(19) United States
(12) Patent Application Publication
(10) Pub. No.: US 2024/0207679 A1
(43) Pub. Date: Jun. 27, 2024

(54) WORKOUT INTENSITY METRICS FOR USERS OF A CONNECTED FITNESS PLATFORM

(71) Applicant: Peloton Interactive, Inc., New York, NY (US)

(72) Inventors: Michelle HESSEL, New York, NY (US); David PACKLES, Brooklyn, NY (US); Bud INTONATO, Brooklyn, NY (US); Marissa VIVORI, New York, NY (US); Nari PARK, Astoria, NY (US); William KRANICH, Brooklyn, NY (US)

(21) Appl. No.: 18/557,756

(22) PCT Filed: Apr. 29, 2022
(86) PCT No.: PCT/US22/26960

§ 371 Date: Oct. 27, 2023

Related U.S. Application Data
(60) Provisional application No. 63/182,361, filed on Apr. 30, 2021.

(51) Int. Cl. A63B 24/00; A63B 71/06; G16H 20/30; G16H 40/67

(52) U.S. Cl. CPC A63B 24/0062; A63B 71/0622; G16H 20/30; G16H 40/67; A63B 2071/065; A63B 2220/05; A63B 2230/065

(57) ABSTRACT

Systems and methods that determine a “strive score,” “effort score,” and/or other metrics that represent intensity levels or effort levels for the user as they perform different workouts or exercise activities are described. For example, the user can perform various activities via exercise machines (e.g., treadmills and/or exercise bicycles) or without machines (e.g., Pilates, running, lifting weights), and the systems and methods can monitor the user's heart rate to determine both specific and overall intensities or efforts expended by the user when performing the activities. The systems and methods can utilize the scoring metrics to present visual graphics or indicia that inform a user of their current effort within an exercise class or activity, their cumulative effort within the class or activity, their comparative effort within the class or activity, and/or other information.

### CROSS REFERENCE TO RELATED APPLICATIONS

This application is a national phase entry of International Application No. PCT/US2022/026960 filed Apr. 29, 2022, which claims priority to U.S. Provisional Application No. 63/182,361, filed on Apr. 30, 2021, entitled DETERMINING WORKOUT INTENSITY FOR A CONNECTED FITNESS PLATFORM, which are hereby incorporated by reference in their entirety.

### BACKGROUND

The world of connected fitness is an ever-expanding one. This world can include a user taking part in an activity (e.g., running, cycling, lifting weights, and so on), other users also performing the activity, and other users doing other activities. The users may be utilizing a fitness machine (e.g., a treadmill, a stationary bike, a strength machine, a stationary rower, and so on), or may be moving through the world on a bicycle or other equipment.

The users can also be performing other activities that do not include an associated machine, such as running, strength training, yoga, stretching, hiking, climbing, and so on. These users can have wearable devices or mobile devices (e.g., heart rate monitors) that monitor the activity or performance of the users. The users can also perform the activity in front of a user interface (e.g., a display or device) presenting content associated with the activity, or outside of any displayed content.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), and so on.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIGS. 2A-2B are diagrams illustrating example user interfaces that present effort score information for a user of an exercise class.

FIG. 3 is a flow diagram illustrating a method for presenting effort information to a user performing an exercise activity during an exercise class.

FIGS. 4A-4D are diagrams illustrating example user interfaces that present effort score information during a presented exercise class.

FIG. 5 is a flow diagram illustrating a method for modifying an exercise class for a user.

FIG. 6 is a flow diagram illustrating a method for recommending exercise classes to a user.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods determine a “strive score,” “effort score,” and/or other metrics that represent intensity levels or effort levels for the user as they perform different workouts or exercise activities. For example, the user can perform various activities via exercise machines (e.g., treadmills and/or exercise bicycles) or without machines (e.g., Pilates, running, lifting weights), and the systems and methods can monitor the user's heart rate to determine both specific and overall intensities or efforts expended by the user when performing the activities.

In some embodiments, the systems and methods can utilize the scoring metrics to present visual graphics or indicia that inform a user of their current effort within an exercise class or activity, their cumulative effort within the class or activity, their comparative effort within the class or activity, and/or other information.

In some embodiments, the systems and methods utilize the scoring or metrics to assess or determine an overall effort performed by a user during an exercise class, such as an instructor-led class, and perform various actions based on the determined effort. For example, the systems and methods can utilize the determines scores or metrics to identify other classes to recommend to the user, to modify an ongoing class, or perform other actions that tailor an exercise class or connected fitness experience for the user, among other benefits.

Various embodiments of the system and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

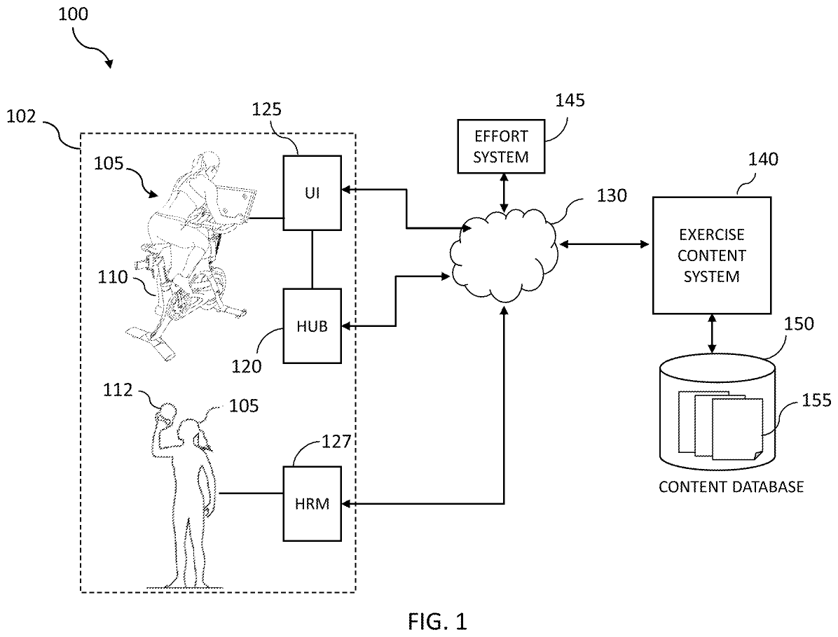

The technology described herein is directed, in some embodiments, to providing a user with an enhanced user experience when performing an exercise activity, such as an exercise activity as part of a connected fitness system or other exercise system. FIG. 1 is a block diagram illustrating a suitable network environment 100 for users of an exercise system.

The network environment 100 includes an activity environment 102, where a user 105 is performing an exercise activity, such as a cycling activity. In some cases, the user 105 can perform the activity with an exercise machine 110, such as exercise bicycle, a treadmill, a rowing machine, a stair climber, and so on. Further, the exercise activity performed by the user 105 can include a variety of different workouts, activities, actions, and/or movements not associated with a machine, such as movements associated with lifting weights 112 (as shown), stretching, doing yoga, Pilates, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., streamed classes, dynamically changing video, audio, video games, instructional content, and so on) to the user 105 during an activity or workout.

The exercise machine 110 includes a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box or other similar device that outputs signals to a display, such as the user interface 125. Thus, the media hub 120 can operate to both capture images of the user 105 during an activity, while also presenting content (e.g., time-based or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

In some cases, a heart rate monitor (HRM) 127 or other wearable device (e.g., smart watch, headphones, fitness trackers, and so on) can capture biometric information about the user 105, such as heart rate, movement information, sleep information, and so on. The HRM 127 can capture the user's heart rate and other information during machine-based activities and/or other activities, such as offline or class-based activities that do not utilize the exercise machine 110. In some cases, the exercise machine can include components configured to capture biometric information for the user 105, such as heart rate information.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) an exercise content system 140 over the network 130. In other cases, the media hub 120 controls the communication of content to/from the exercise content system 140 over the network 130 and presents the content to the user via the user interface 125.

The exercise content system 140, located at one or more servers remote from the user 105, can include various content libraries (e.g., classes, movements, tutorials, and so on) and perform functions to stream or otherwise send content to the machine 110, the media hub 120, and/or the user interface 125 over the network 130.

A content database 150 stores content 155 (e.g., video files or image files) that presents a pre-recorded class to a user. The content can include images, video, and other visual information that present the class, music and other audio information to be played during the activity, and various overlay or augmentation information that is presented along with the audio/video content. Further, the database 150 can include various content libraries (e.g., classes, movements, tutorials, and so on) associated with the content presented to the user during a selected experience.

Thus, in some embodiments, the exercise content system 140 can build or modify classes by combining different segments or movements of classes to dynamically generate a class for a user or modify a class within which the user is performing an exercise activity (e.g., the user has started an archived class). The system 140 can insert segments into an ongoing class, add segments to an ongoing class, or switch the user to another class midway through or during the initial class (e.g., at a point or time interval when the user is moved out of the initial class).

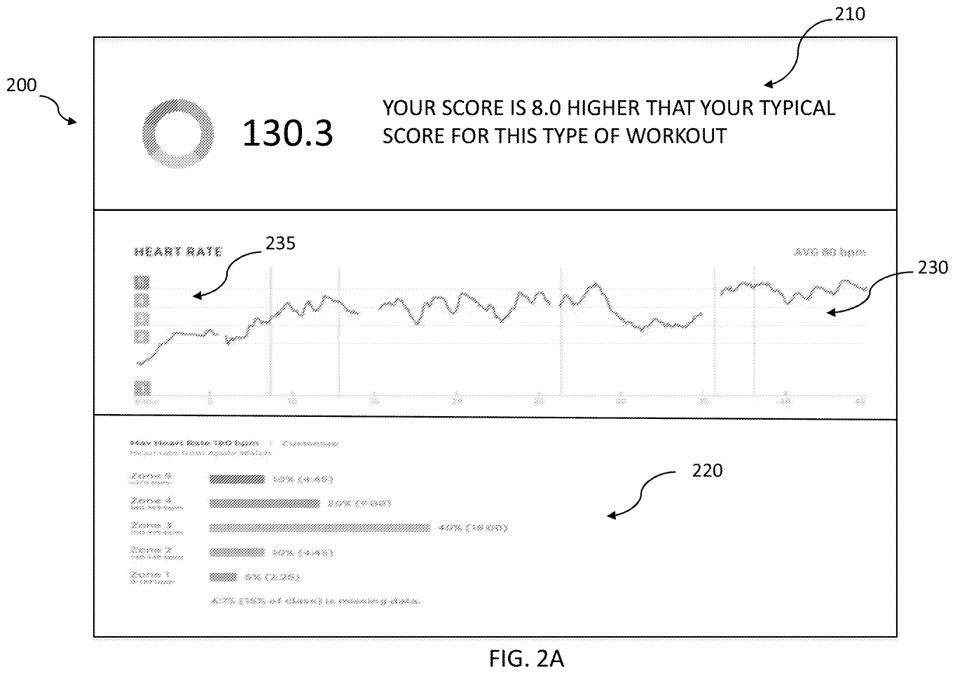

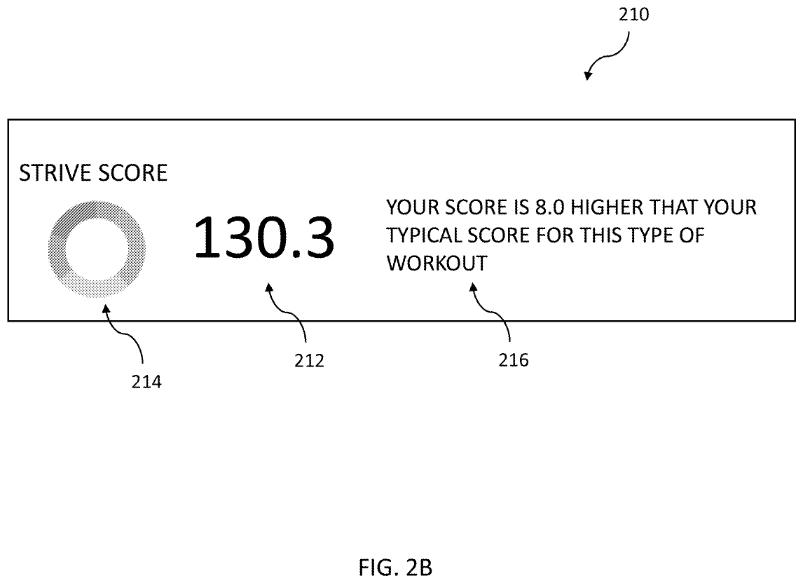

As described herein, an effort system 145 can include various components configured to access biometric information about the user 105 and determine various metrics or scores for the user that represent the user's performance effort or performance intensity during exercise activities. The effort system 145, for example, can receive heart rate information from the heart rate monitor 127 for the user during various activities (e.g., a cycling class, a stretching class, a lifting class, and an offline, outdoor run), and determine an effort score or strive score for each class (and for some or all classes combined). Further details regarding the functionality of the effort system 145, including user interfaces presented by the effort system 145, are described with respect to FIGS. 2-12

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 130 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 130 and are shown as separate connections, these connections can be any kind of local, wide area, wired, or wireless network, public or private.

Further, any or all components depicted in the Figures described herein can be supported and/or implemented via one or more computing systems or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of Determining Workout Intensities for Users

As described herein, in some embodiments, the systems and methods, via the effort system 145, perform various methods or operations to determine workout intensities, represented by various scores or metrics (e.g., an effort score or strive score), for user workouts or other exercise activities.

In some cases, the systems and methods can utilize these scores or metrics to present different types of information to the users, to perform actions based on the scores/metrics (e.g., recommend classes, recommend other types of activities, modify classes, provide achievements, and so on). Further, the systems and methods, via the effort system 145, can track a user over different exercise activities, including activities tied to an exercise machine (e.g., the exercise machine 110) and activities not tied to a machine.

As described herein, the effort system 145 can generate or determine a score or metric for a user's participation in an exercise class, such as an instructor-led class that includes a cardio-based activity, a strength-based activity, and so on. FIG. 2A depicts an example user interface 200 presenting a workout history for a user that has completed a workout or exercise class. The workout history presents various information about the class, including effort score information 210, heart rate information 220 for the entire class, and heart rate zone information, which is utilized by the effort system 145 determine an effort score (or strive score) for the workout, class, or activity.

For example, the effort system 145 can assign heart rate values to multiple heart rate zones (e.g., zones 1-5) 230, such as a “Zone 1” for heart rate values between 0-139 bpm (beats per minute), a “Zone 2” for heart rate values between 140-149 bpm, a “Zone 3” for heart rate values between 150-159 bpm, a “Zone 4” for heart rate values between 160-169 bpm, and a “Zone 5” for heart rate values between of 170 bpm or above.

The effort system 145, as depicted in FIG. 2A, can track the percentage of the class in which the user was in the various heart rate zones, and determine the scores/metrics based on various rules or formulas that generate a score or metric using the zone information. For example, the user is assigned a strive score of 130.3 for the workout, determined based on the heart rate of the user being within the different heart rate zones for different time durations (or percentages) of the class.

FIG. 2B depicts details about the effort score information 210. The effort score information 210 can be presented within a streaming class (e.g., a pre-recorded, archived, or live class) and/or with workout activity information for a completed class. The effort score information 210 can include different color zones 214 that indicate the heart rate zones (and associated time periods or relative percentages) achieved by the user during the activity, an overall score 212 for the workout, context information 216 for the workout, and so on.

As described herein, while output is an objective, comparative metric that drives leaderboards and is based on exercise machine information (e.g., cadence and resistance for bikes and/or incline and speed for treadmills), the effort score or strive score is personal and non-competitive, and based on the heart rate (or, optionally, other biometric information) of the user during activities.

Thus, in some cases, the user interface 200 can present information that shows output and effort score in combination, informing the user how output changes with respect to performed effort, perhaps indicating a user is getting stronger or more fit over time. For example, the user interface 200 can present the effort score information 210, which is based on historical effort scores for the user and compare that to an expected output for the class.

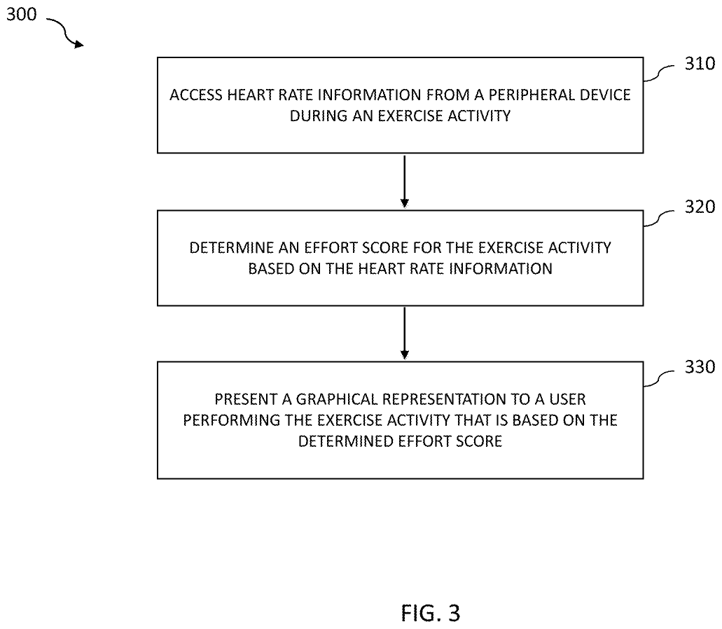

FIG. 3 is a flow diagram illustrating a method 300 for presenting effort information to a user performing an exercise activity during an exercise class. The method 300 may be performed by the effort system 145 and, accordingly, is described herein merely by way of reference thereto. It will be appreciated that the method 300 may be performed on any suitable hardware.

In operation 310, the effort system 145 accesses information associated with a heart rate of a user of an exercise activity performed by the user. For example, the effort system 145 can receive or access information from a heart rate monitor or other peripheral device worn by the user that tracks the heart rate of the user during the exercise activity. As described herein, the user can be performing the exercise activity as part of an exercise class, such as a streamed exercise class displayed to the user via a display device, exercise machine, mobile device, smart watch, and so on.

In operation 320, the effort system 145 determines a metric that represents an overall effort of the user when performing the exercise activity. For example, the effort system 145 can determine or calculate a score that is based on the heart rate of the user being within one or more heart rate zones of multiple different heart rate zones that define a range of heart rate values exhibited by the user when the user is performing the exercise activity.

In some cases, the effort system 145 can determine the score (e.g., the effort score or strive score) by determining, for a duration of an exercise class, time spent by the user within multiple different heart rate zones (where each heart rate zone of the multiple different heart rate zones identifies a range of heart rate values for the user), assign a different score multiplier to each heart rate zone of the multiple different heart rate zones, and generating a score based on the time spent by the user within the multiple different heart rate zones and the assigned score multipliers to each of the heart rate zones of the multiple different heart rate zones.

For example, the effort system 145 determines, for an entire duration of the exercise class, a percentage of the entire duration in which the heart rate of the user was in at least two different heart rate zones and generates the metric as a cumulative score based on the determined percentages for each of at least two different heart rate zones.

In some cases, the effort system 145 determines the effort score or metric based on time spent in each heart rate zone, with higher zones earning more points or having higher or greater assigned weights. For example, a user can earn a fraction of a point for each second that they are in a heart rate zone, which is calculated by a zone ratio and point multiplier.

An example determination is as follows:
Zone 1=(0.1*3)/60 points/second, and Zone 2=(0.2*3)/60 points/second.

Further, an example zone ratio is as follows:
Zone Ratio: Zone 1=1×, Zone 2=2×, Zone 3=4×, Zones 4 or 5=8×

Of course, other zones, ratios, multipliers, and other factors can be utilized when determining the effort score. Further, in some cases, the systems and methods can utilize different zone ratios or multipliers based on a variety of factors, such as the type of user, the type of activity being performed by the user, the resting heart rate for the user, and so on.

For example, the effort system 145 can determine an effort that is based on a comparison of the user's heart rate to a baseline heart rate (e.g., their heart rate when starting an exercise activity), their resting or average heart rate, and so on. Further, the effort system 145 can utilize a scoring mechanism or formula that is specific to a user, their experience level for an activity or class, the difficulty level assigned to the class, and so on.

For example, the effort system 145 can determine an effort score for a user new to a certain activity (e.g., a user new to rowing) that has fewer zones, because the user effort is going to be focused on performing the activity properly, instead of achieving a max effort or intensity. As another example, the effort system can determine an effort score for a user of an interval class (e.g., a HIIT class) that has a unique set of multipliers for the higher heart rate zones, because the user is focused on performing at max effort, and having the score reflect the effort.

Thus, the effort system 145 can tailor or modify a determination of an effort score for a user that is based on the user type and/or class type, among other factors.

In operation 330, the effort system 145 presents a graphic to the user via a display that is associated with the metric representing the overall effort of the user when performing the exercise activity (or when completing the exercise activity). For example, the effort system 145, via the user interfaces described herein, can present a visual element that identifies a duration in which a value for the heart rate of the user was within one or more heart rate zones of the multiple different heart rate zones that define a range of heart rate values and/or the total effort score assigned to the user during the exercise activity.

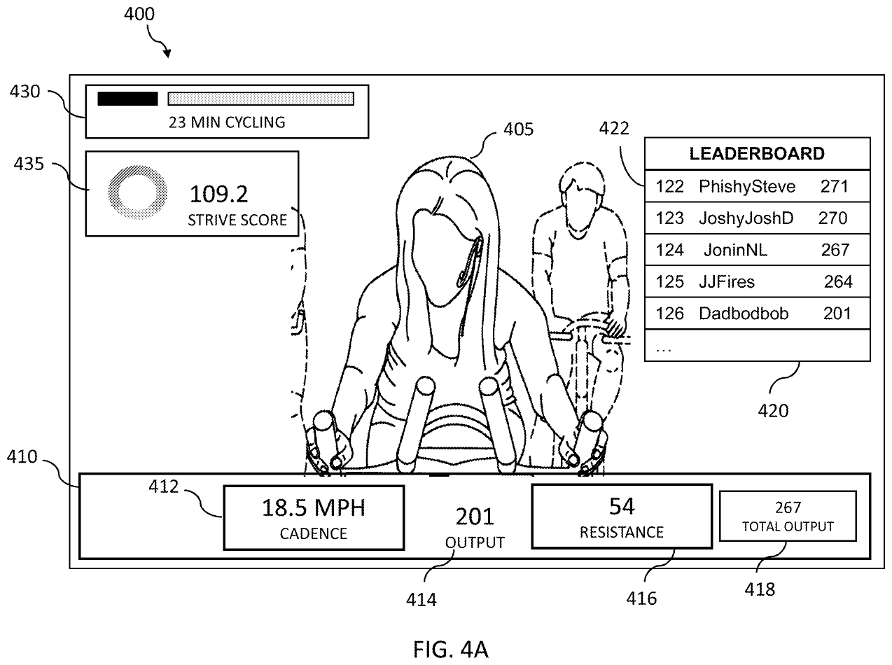

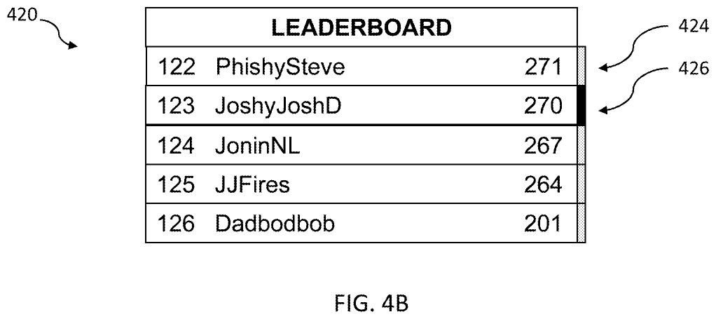

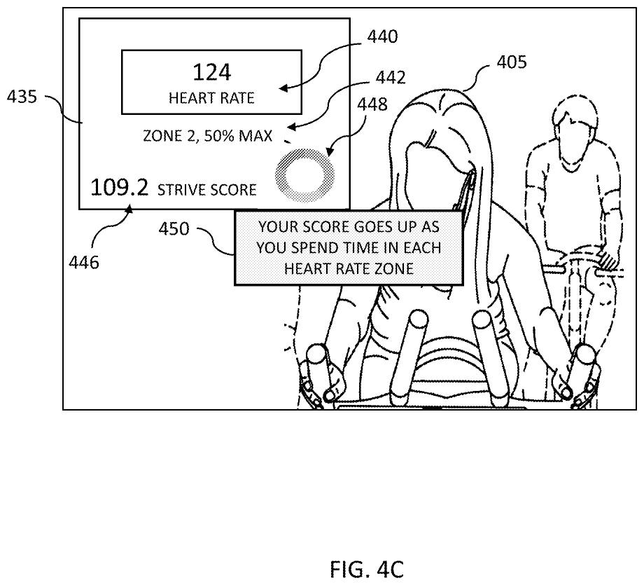

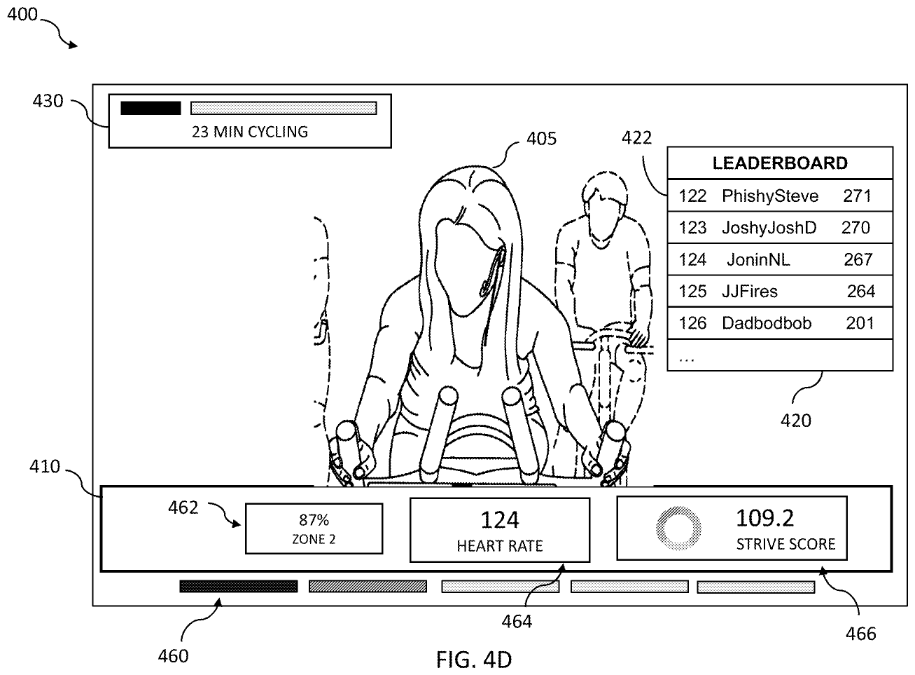

In some cases, the effort system 145 can cause the visual display of an exercise class (e.g., an instructor-led class) to present effort score information. FIGS. 4A-4D are diagrams illustrating example user interfaces that present effort score information during a presented exercise class.

FIG. 4A depicts a user interface 400 that presents an exercise class, such as a cycling class led by an instructor 405. The class UI 400 includes a display 410 of various class metrics, including a cadence metric 412, a current output metric 414, a resistance metric 416, a total output metric 418, and so on.

Further, the UI 400 can include a leaderboard 220, which displays a user “PhishySteve” 422 in the context of other users of the class. In general, the leaderboard 420 can display the relative performance of all users or one or more subgroups of users. For example, the user 422 may be able to select a leaderboard that shows the performance of users in a particular age group, male users, female users, male users in a particular age group, users in a particular geographic area, and so on. Users may be provided with the ability to deselect the leaderboard entirely and remove it from the screen.

Further, the leaderboard can incorporate various social networking aspects, such as allowing the user to follow other users, or to create groups or circles of users. Thus, user lists and information may be accessed, sorted, filtered, and used in a wide range of different ways. For example, other users can be sorted, grouped and/or classified based on any characteristic including personal information such as age, gender, weight, or based on performance such as current power output, speed, effort score, or a custom score.

The leaderboard 420 can be fully interactive, allowing the user 422 to scroll up and down through user rankings, and to select a user to access their detailed performance data, create a connection (such as choosing to follow that user), or establish direct communication (such as through an audio and/or video connection). The leaderboard 420 can also display the user's personal best performance in the same or a comparable class, to allow the user to compare their current performance to their previous personal best. The best performance information can include output, effort score, and so on. The leaderboard 420 can also highlight certain users, such as those that the user follows, or provide other visual cues to indicate a connection or provide other information about a particular entry on the leaderboard.

In some cases, the leaderboard 420 can also allow the user to always view their position and performance information while scrolling through the leaderboard. For example, when the user scrolls up toward the top of the leaderboard 420 (such as by dragging their fingers upward on a touchscreen display presenting the leaderboard 420), when the user's window reaches the bottom of the leaderboard, it will lock in position and the rest of the leaderboard will scroll underneath it. Similarly, when the user scrolls down toward the bottom of the leaderboard, when the user's window reaches the top of the leaderboard, it will lock in position and the rest of the leaderboard will continue to scroll underneath it.

FIG. 4B depicts additional details about the leaderboard 420. The leaderboard 420 can be a dynamic updating ranked list of the users of the class, which lists the users based on their total output or other comparative metrics. In some cases, the leaderboard 420 can present effort score information for other users of the class, such as via different color bars 424, 426 within the individual user entries displayed by the leaderboard 720.

For example, the color bar 424 can indicate that the user “PhishySteve” 422 is performing in Zone 3 (e.g., moderate effort), while the color bar 426 indicates that a different user, “JoshyJoshD,” is performing at maximum effort (in Zone 5). The color bars 424, 426, therefore, can indicate the effort levels of some or all users of the class at any given point in time or segment with the class.

Referring back to FIG. 4A, the UI 400 includes a timeline element 430, which displays the progress of a user within the exercise class presented by the instructor 405, and an effort score element 435, which presents the effort score information and other related information for the user during the exercise class.

FIG. 4C depicts additional details about the effort score element 435. The effort score element 435 can include heart rate information 440 that is based on the user's current heart rate (measured by an associated heart rate monitor) as well as zone information 442 that identifies the heart rate zone within which the user is currently performing.

The element 435 also includes effort score information, such as a current effort score 446 for the exercise class, and a graphic 448 (e.g., like the color zone 214), which indicates the heart rate zones (and associated time periods or relative percentages) within which the user has performed during the class. The effort score information, therefore, can display heart rate information, zone information, a running effort score calculation, and so on. The information can be presented as numbers, colors, filled circles or lines, and so on.

In some cases, the effort score information is associated with a dialog box 450, which displays information about the effort score information 435. The dialog box 450 can present information that identifies how the effort score is calculated or determined. For example, the box 450 can include an effort score, a zone within which the user is exercising, and other information associated with the intensity of the workout, such as how the effort score is determined during the workout to provide the user with guidance for increasing, maintaining, and/or lowering their score during the workout.

FIG. 4D depicts the class UI 400 where the effort score information is located with the metrics bar 410 or display. The metrics bar 410 can present the zone information 462, current heart rate information 464, and strive score information 466. Further, the UI 400 can include visual indicia 460 that depicts the zone the user 422 is currently exercising (e.g., Zone 1 is filled in and the user 422 is exercising in Zone 2, of five zones).

Thus, the UIs described herein can display various types of information or graphics, including effort or strive score information for a class, strive scores within class leaderboards, strive score modules that reflect a user's current intensity or effort, strive scores within scorecards for classes, onboarding or tutorial information, workout summaries that include strive scores, strive scores and benchmarks within workout histories, and/or aggregate or total strive scores in user overview information.

Further, the UIs can be displayed by various devices, such as mobile devices, televisions or set-top box displays, web/digital interfaces, smart watches, laptops, exercise machine displays, and so on.

### Examples of Performing Actions Based on Effort Scores

As described herein, in some embodiments, the systems and methods utilize the scoring or metrics to assess or determine an overall effort performed by a user during an exercise class, such as an instructor-led class, and perform various actions based on the determined effort scores.

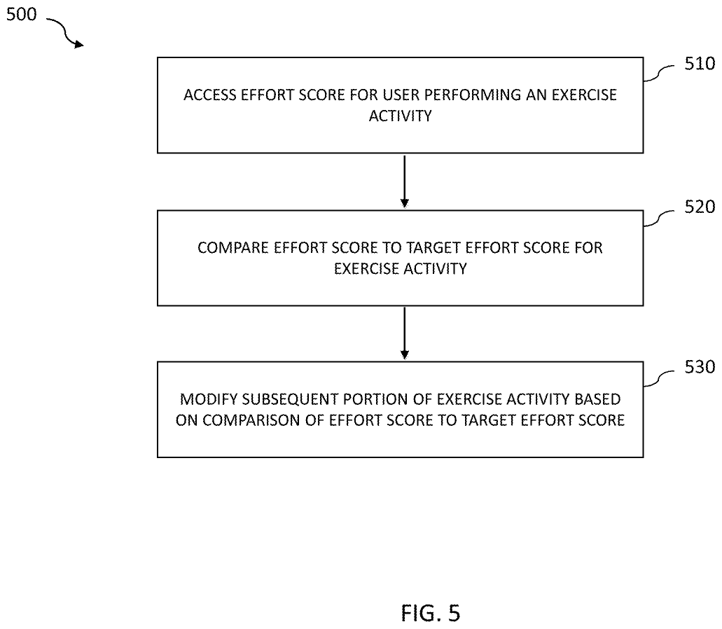

FIG. 5 is a flow diagram illustrating a method 500 for modifying an exercise class for a user. The method 500 may be performed by the effort system 145 and, accordingly, is described herein merely by way of reference thereto. It will be appreciated that the method 500 may be performed on any suitable hardware.

In operation 510, the effort system 145 accesses an effort score for a user performing an exercise activity. For example, the effort system 145 can access an effort score being calculated (as described herein) during an exercise class, such as an instructor-led class.

In operation 520, the effort system 145 determines that an effort score for a user performing an exercise activity during an exercise class is above a target effort score for the streamed exercise class. For example, the effort system 145 can determine the user is above a threshold or target effort score for all users of the class or other users like the user.

For example, a class having a difficulty level of 7/10, with a 20 minute duration, can have a target effort score of 100, whereas a similar class having a greater difficulty level of 8/10 can have a higher target effort score (e.g., 110).

As another example, the system 145 can identify or define the target effort score as being based on a user's previous effort scores. The system 145 can determine an average (e.g., weighted average) of previous scores (e.g., the most recent scores) and use that average score as the target score.

In operation 530, the effort system 145 modifies a subsequent segment of the streamed exercise class based on the effort score determined for the user being above the target effort score for the streamed exercise class. For example, the effort system 145 can dynamically modify the class to insert an additional segment, to modify a subsequent segment, or to otherwise increase or decrease an intensity or difficulty level for the exercise class.

Thus, when the user is below the target effort score, the system 145 can determine the user is not being challenged by the class, and modify the class to increase the difficulty level, move the user to another class (at a similar segment or time interval within the new class), insert an additional segment, and so on.

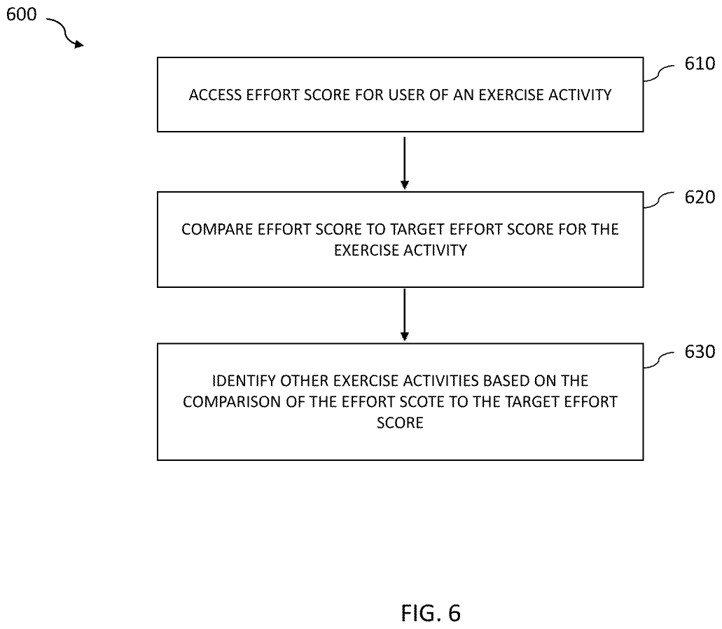

In some embodiments, the effort system 145 can act as a recommendation engine or system that utilizes effort scores to identify, select, filter, or recommend classes or activities to users. FIG. 6 is a flow diagram illustrating a method 600 for recommending exercise classes to a user.

In operation 610, the effort system 145 accesses an effort score for a user that completed an exercise activity or class. For example, the effort system 145 can access an effort score being calculated (as described herein) for an exercise class, such as an instructor-led class, and/or can access a user's workout history and identify an effort score or scores earned during one or more exercise classes. In some cases, the effort system 145 determines a metric that represents an overall effort of the user when performing an exercise activity during a streamed exercise class based on information associated with a heart rate of the user, as described herein.

In some cases, the effort system 145 assesses a user's workout history and identifies trends or cumulative effort scores to determine a metric that represents a cumulative effort score for the predetermined period of time when the user performed the certain exercises.

In operation 620, the effort system 145 compares the determined metric that represents the overall effort of the user to a target metric for the streamed exercise class. The effort system 145 can determine the metric that represents an overall effort of the user is below the target metric for the streamed exercise class and select an exercise class having a difficulty level greater than a difficulty level assigned to the steamed exercise class for recommendation to the user. Conversely, the effort system 145 can determine the metric that represents an overall effort of the user is above the target metric for the streamed exercise class and select an exercise class having a difficulty level lower or less than a difficulty level assigned to the steamed exercise class for recommendation to the user.

In some cases, the effort system 145 can combine the user's effort score and class rank (or percentage) to determine whether a class was above, below, and/or at an effort or fitness level of the user. For example, the user participates in a floor-based strength class and ends the class ranked in the top 10 percent, with an effort score that is within the bottom 50 percent of the class (or below an average effort score for the user). The system 145, using such information, can recommend future classes to the user that have difficulty levels above the difficultly level assigned to the class.

In operation 630, the effort system 145 presents a recommendation for another streamed exercise class that is based on the comparison of the determined metric to the target metric for the streamed exercise class. For example, the effort system 145 can cause a connected fitness system to present classes that are appropriate to the user based on the comparison of effort scores and/or filter a list of classes to identify classes that have difficulty levels appropriate for the user's level of fitness or effort.

Thus, the effort system 145, in some cases, can utilize effort scores for a user to tailor a list of exercise classes to present to the user, such as exercise classes that are based on or otherwise match the user's effort scores or predicted future effort scores, among other benefits. Further, the effort system 145, using the information about effort scores for the user, can inform a user about their fitness and perform actions that are based on tracking an increasing fitness level (or, decreasing fitness level) for the user, among other benefits.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system, comprising:
a processor; and
one or more computing memories coupled to the processor, wherein the processor is configured to:
access information associated with a heart rate of a user of an exercise activity performed by the user during an exercise class presented to the user via a display;
determine a metric that represents an overall effort of the user when performing the exercise activity during the exercise class based on the information associated with the heart rate of the user; and
present a graphic to the user via the display that is associated with the metric representing the overall effort of the user when performing the exercise activity.

2. The system of claim 1, wherein the processor determines the metric that represents an overall effort of the user when performing the exercise activity by:
determining, for a duration of the streamed exercise class, time spent by the user within multiple different heart rate zones,
wherein each heart rate zone of the multiple different heart rate zones identifies a range of heart rate values for the user;
assigning a different score multiplier to each heart rate zone of the multiple different heart rate zones; and
generating a score based on the time spent time spent by the user within multiple different heart rate zones and the assigned score multipliers to each of the heart rate zones of the multiple different heart rate zones.

3. The system of claim 1, wherein the processor determines the metric that represents an overall effort of the user when performing the exercise activity as a score that is based on the heart rate of the user being one or more heart rate zones of multiple different heart rate zones that define a range of heart rate values exhibited by the user when the user is performing the exercise activity.

4. The system of claim 1, wherein the processor presents a score determined for the user when the user is performing the exercise activity.

5. The system of claim 1, wherein the processor presents a visual element that identifies a duration in which a value for the heart rate of the user was within one or more heart rate zones of multiple different heart rate zones that define a range of heart rate values.

6. The system of claim 1, wherein the processor determines the metric that represents an overall effort of the user when performing the exercise activity by:
determining, for an entire duration of the exercise class, a percentage of the entire duration in which the heart rate of the user was in at least two different heart rate zones; and
generating the metric as a cumulative score based on the determined percentages for each of at least two different heart rate zones.

7. The system of claim 1, wherein the processor accesses information associated with a heart rate of a user from information received from a peripheral device worn by the user when performing the exercise activity.

8. The system of claim 1, wherein the metric that represents an overall effort of the user when performing the exercise activity is based on:
an amount of time of the exercise class within which a value of the heart rate of the user was within one or more heart rate zones; and
a resting heart rate for the user.

9. The system of claim 1, wherein the metric that represents an overall effort of the user when performing the exercise activity is based on:
an amount of time of the exercise class within which a value of the heart rate of the user was within one or more heart rate zones; and
the type of exercise activity performed by the user.

10. The system of claim 1, wherein the metric that represents an overall effort of the user when performing the exercise activity is based on:
an amount of time of the exercise class within which a value of the heart rate of the user was within one or more heart rate zones; and
an experience level for the user in performing the exercise activity.

11. The system of claim 1, wherein the exercise activity is a running activity performed by the user on a treadmill.

12. The system of claim 1, wherein the exercise activity is a cycling activity performed by the user on an exercise bicycle.

13. The system of claim 1, wherein the exercise activity is a rowing activity performed by the user on a rowing machine.

14. The system of claim 1, wherein the exercise activity is a lifting activity performed by the user.

15. The system of claim 1, wherein the exercise activity is a cardio-based activity presented to the user via a display associated with the user.

16. A method for recommending an exercise class to a user, the method comprising:
determining a metric that represents an overall effort of the user when performing an exercise activity during an exercise class based on information associated with a heart rate of the user; and
comparing the determined metric that represents the overall effort of the user to a target metric for the exercise class; and
presenting a recommendation for another exercise class that is based on the comparison of the determined metric to the target metric for the exercise class.

17. The method of claim 16, wherein comparing the determined metric that represents the overall effort of the user to a target metric for the exercise class includes:
determining the metric that represents an overall effort of the user is below the target metric for the exercise class; and
selecting an exercise class having a difficulty level greater than a difficulty level assigned to the exercise class for recommendation to the user.

18. The method of claim 16, wherein determining a metric that represents an overall effort of the user when performing an exercise activity during an exercise class includes:
determining, for a duration of the exercise class, time spent by the user within multiple different heart rate zones,
wherein each heart rate zone of the multiple different heart rate zones identifies a range of heart rate values for the user;
assigning a different score multiplier to each heart rate zone of the multiple different heart rate zones; and
generating a score based on the time spent time spent by the user within multiple different heart rate zones and the assigned score multipliers to each of the heart rate zones of the multiple different heart rate zones.

19. A non-transitory computer-readable medium whose contents, when executed by a computing system, cause the computing system to perform a method, the method comprising:
determining an effort score for a user performing an exercise activity during a streamed exercise class is above a target effort score for the streamed exercise class; and
modifying a subsequent segment of the streamed exercise class based on the effort score determined for the user being above the target effort score for the streamed exercise class.

20. The non-transitory computer-readable medium of claim 19, wherein the effort score for the user performing an exercise activity during a streamed exercise class is based on:
determining, for a duration of the streamed exercise class, time spent by the user within multiple different heart rate zones,
wherein each heart rate zone of the multiple different heart rate zones identifies a range of heart rate values for the user; and
generating a score based on the time spent time spent by the user within multiple different heart rate zones.
